(19) United States
(12) Patent Application Publication
(10) Pub. No.: US 2023/0397840 A1
(43) Pub. Date: Dec. 14, 2023

(54) DETECTION DEVICE, DETECTION SYSTEM, DETECTION METHOD, AND PROGRAM RECORDING MEDIUM

(71) Applicant: NEC Corporation, Minato-ku, Tokyo (JP)

(72) Inventors: Chenhui HUANG, Tokyo (JP); Kenichiro FUKUSHI, Tokyo (JP); Zhenwei WANG, Tokyo (JP); Fumiyuki NIHEY, Tokyo (JP); Hiroshi OKUDA, Tokyo (JP)

(73) Assignee: NEC Corporation, Minato-ku, Tokyo (JP)

(21) Appl. No.: 18/033,471

(22) PCT Filed: Nov. 10, 2020
(86) PCT No.: PCT/JP2020/041897

§ 371 Date: Apr. 24, 2023

(51) Int. Cl. A61B 5/11; A61B 5/00

(52) U.S. Cl. CPC A61B 5/112; A61B 5/1114; A61B 5/1121; A61B 5/6807; A61B 2562/0219

(57) ABSTRACT

A detection device includes a waveform generation unit that generates a gait waveform using sensor data related to a motion of a foot, and a detection unit that detects a gait event from the gait waveform based on a condition set for each of an angle, an angular velocity, and an acceleration in a sagittal plane.

### TECHNICAL FIELD

The present disclosure relates to a detection device and the like that detect a gait event.

### BACKGROUND ART

With increasing interest in healthcare that performs physical condition management, a service in which a gait including a gait feature is measured and that provides information related to the gait to a user has attracted attention. When a gait event such as an event in which the heel touches the ground (also referred to as heel strike) and an event in which the toe leaves the ground (also referred to as toe off) can be detected from the data regarding gait, a service according to the gait can be more accurately provided. For example, when a gait event of a person with physical disabilities can be detected in the same manner as a healthy person, a service according to the gait can be provided to more people.

PTL 1 discloses a gait characteristic evaluation system capable of measuring a three-dimensional gait characteristic of a person who is difficult to walk for a long time. The system of PTL 1 performs an arithmetic process on data such as an acceleration or an angular velocity measured by a sensor mounted on the toe of the foot to generate a three-dimensional locus of the toe of each step. The system of PTL 1 derives three-dimensional gait characteristics such as the number of steps, a stride, a cadence, a gait speed, a distance between a foot toe and a gait surface, and a swinging angle of the foot toe from the generated three-dimensional locus.

### CITATION LIST

### Patent Literature

PTL 1: JP 2010-110399 A

### SUMMARY OF INVENTION

### Technical Problem

The system of PTL 1 can derive three-dimensional gait characteristics such as the number of steps, a stride, a cadence, a gait speed, a distance between a foot toe and a gait surface, and a swinging angle of the foot toe with respect to a person who is difficult to walk. However, the system of PTL 1 can verify the behavior of the foot with respect to a person who is difficult to walk but cannot detect a gait event such as heel strike or toe off.

An object of the present disclosure is to provide a detection device and the like capable of detecting a gait event based on a gait waveform even regarding a gait of a person with physical disabilities.

### Solution to Problem

A detection device according to an aspect of the present disclosure includes a waveform generation unit that generates a gait waveform using sensor data related to a motion of a foot, and a detection unit that detects a gait event from the gait waveform based on a condition set for each of an angle, an angular velocity, and an acceleration in a sagittal plane.

In a detection method executed by a computer according to an aspect of the present disclosure, the method includes generating a gait waveform using sensor data related to a motion of a foot, and detecting a gait event from the gait waveform based on a first condition, a second condition, and a third condition set for an angle, an angular velocity, and an acceleration, respectively, in a sagittal plane.

A program according to an aspect of the present disclosure causes a computer to execute the steps of generating a gait waveform using sensor data related to a motion of a foot, and detecting a gait event from the gait waveform based on a first condition, a second condition, and a to third condition set for an angle, an angular velocity, and an acceleration, respectively, in the sagittal plane.

### Advantageous Effects of Invention

According to the present disclosure, it is possible to provide a detection device and the like capable of detecting a gait event based on a gait waveform even regarding a gait of a person with physical disabilities.

### BRIEF DESCRIPTION OF DRAWINGS

FIG. 1 is a block diagram illustrating an example of a configuration of a detection system according to a first example embodiment.

FIG. 2 is a conceptual diagram illustrating an arrangement example of a data acquisition device of the detection system according to the first example embodiment.

FIG. 3 is a conceptual diagram for explaining a coordinate system set in the data acquisition device of the detection system according to the first example embodiment.

FIG. 4 is a conceptual diagram for explaining a human body surface applied to a detection device of the detection system according to the first example embodiment.

FIG. 5 is a conceptual diagram for explaining a gait event.

FIG. 6 is an example of a gait waveform generated by the detection device of the detection system according to the first example embodiment.

FIG. 7 is another example of the gait waveform generated by the detection device of the detection system according to the first example embodiment.

FIG. 8 is a block diagram illustrating an example of a configuration of the data acquisition device of the detection system according to the first example embodiment.

FIG. 9 is a block diagram illustrating an example of a configuration of the detection device of the detection system according to the first example embodiment.

FIG. 10 is a conceptual diagram for explaining detection of a gait event by the detection device of the detection system according to the first example embodiment.

FIG. 11 is a conceptual diagram for explaining an example of detection of a peak by the detection device of the detection system according to the first example embodiment.

FIG. 12 is a conceptual diagram for explaining a detection example and a determination example of a peak by the detection device of the detection system according to the first example embodiment.

FIG. 13 is a conceptual diagram for explaining an example of peak determination by the detection device of the detection system according to the first example embodiment.

FIG. 14 is a flowchart for explaining an example of an outline of an operation of the detection device of the detection system according to the first example embodiment.

FIG. 15 is a flowchart for explaining an example of detection processing by the detection device of the detection system according to the first example embodiment.

FIG. 16 is a block diagram illustrating an example of a configuration of a detection system according to a second example embodiment.

FIG. 17 is a block diagram illustrating an example of a configuration of a detection device of the detection system according to the second example embodiment.

FIG. 18 is a conceptual diagram for explaining an example of a gait state determined using the related art.

FIG. 19 is a conceptual diagram for explaining an example of the gait state determined by a determination unit of the detection system according to the second example embodiment.

FIG. 20 is a block diagram illustrating an example of a configuration of a detection device according to a third example embodiment.

FIG. 21 is a block diagram illustrating an example of a hardware configuration for achieving the detection device according to each example embodiment.

### EXAMPLE EMBODIMENT

Hereinafter, embodiments of the present invention will be described with reference to the drawings. However, the example embodiments described below have technically preferable limitations for carrying out the present invention, but the scope of the invention is not limited to the following. In all the drawings used in the following description of the example embodiment, the same reference numerals are given to the same parts unless there is a particular reason. In the following example embodiments, repeated description of similar configurations and operations may be omitted.

### First Example Embodiment

First, a detection system according to a first example embodiment will be described with reference to the drawings. The detection system of the present example embodiment detects a gait event of a pedestrian using sensor data acquired by a sensor installed on a foot portion of the pedestrian. For example, a gait event includes a timing at which plantarflexion/dorsiflexion of the foot is maximized. For example, the gait event includes an event in which the foot lands on the ground (also referred to as heel strike), an event in which the foot leaves the ground (also referred to as toe off), and the like. Details of the gait event detected by the detection system of the present example embodiment will be described later.

### Configuration

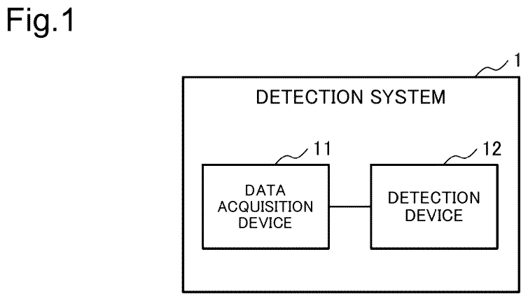

FIG. 1 is a block diagram illustrating a configuration of a detection system 1 of the present example embodiment. The detection system 1 includes a data acquisition device 11 and a detection device 12. The data acquisition device 11 and the detection device 12 may be connected by wire or wirelessly. The data acquisition device 11 and the detection device 12 may be configured by a single device. The detection system 1 may be configured only by the detection device 12 excluding the data acquisition device 11 from the configuration of the detection system 1.

For example, the data acquisition device 11 is installed in footwear such as shoes. In the present example embodiment, an example in which the data acquisition device 11 is disposed at a position corresponding to the back side of the arch of the foot will be described. The data acquisition device 11 includes an acceleration sensor and an angular velocity sensor. Data acquisition device 11 measures a physical quantity related to the motion of the foot such as the spatial acceleration and the spatial angular velocity as the physical quantity related to the motion of the foot of the user wearing the footwear. The physical quantity related to the motion of the foot measured by the data acquisition device 11 includes not only the acceleration and the angular velocity but also the velocity and the angle calculated by integrating the acceleration and the angular velocity. The physical quantity related to the motion of the foot measured by the data acquisition device 11 also includes a position (locus) calculated by second-order integration of acceleration.

The data acquisition device 11 converts the measured physical quantity into digital data (also referred to as sensor data). The data acquisition device 11 transmits the converted sensor data to the detection device 12. For example, the data acquisition device 11 is connected to the detection device 12 via a mobile terminal (not illustrated) carried by the user. The mobile terminal (not illustrated) is a communication device that can be carried by a user. For example, the mobile terminal is a portable communication device having a communication function, such as a smartphone, a smart watch, or a mobile phone. The mobile terminal receives, from the data acquisition device 11, sensor data related to the motion of the user's foot. The mobile terminal transmits the received sensor data to a server or the like on which the detection device 12 is mounted. The function of the detection device 12 may be achieved by an application installed in the mobile terminal. In this case, the mobile terminal processes the received sensor data by application software or the like installed therein.

The data acquisition device 11 is achieved by, for example, an inertial measurement device including an acceleration sensor and an angular velocity sensor. An example of the inertial measurement device is an inertial measurement unit (IMU). The IMU includes a three-axis acceleration sensor and a three-axis angular velocity sensor. Examples of the inertial measurement device include a vertical gyro (VG), an attitude heading (AHRS), and a global positioning system/inertial navigation system (GPS/INS).

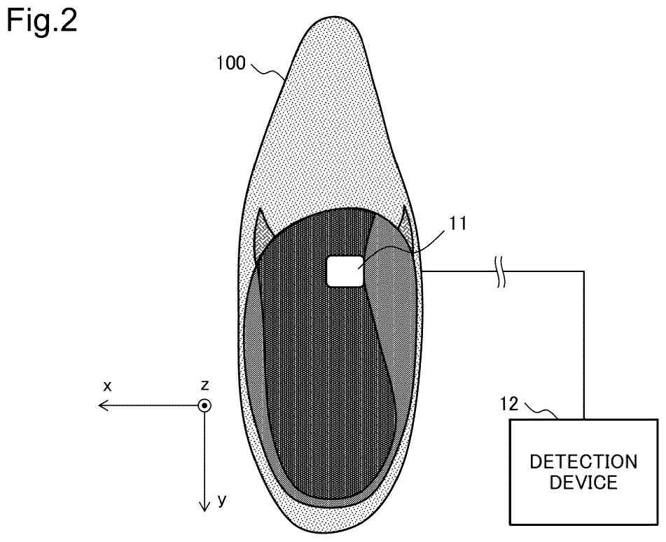

FIG. 2 is a conceptual diagram illustrating an example in which the data acquisition device 11 is installed in a shoe 100. In the example of FIG. 2, the data acquisition device 11 is installed at a position corresponding to the back side of the arch of the foot. For example, the data acquisition device 11 is installed in an insole inserted into the shoe 100. For example, the data acquisition device 11 is installed on the bottom face of the shoe 100. For example, the data acquisition device 11 is embedded in the main body of the shoe 100. The data acquisition device 11 may be detachable from the shoe 100 or may not be detachable from the shoe 100. The data acquisition device 11 may be installed at a position not corresponding to the back side of the arch of the foot as long as it can acquire sensor data regarding the motion of the foot. The data acquisition device 11 may be installed on a sock worn by the user or a decorative article such as an anklet worn by the user. The data acquisition device 11 may be directly attached to the foot or may be embedded in the foot. FIG. 2 illustrates an example in which the data acquisition device 11 is installed in the shoe 100 of the left foot. The data acquisition device 11 is only required to be installed on at least one foot, and may be installed on both left and right feet. When the data acquisition device 11 is installed in the shoes 100 of both feet, the gait event can be detected in association with the motion of both feet.

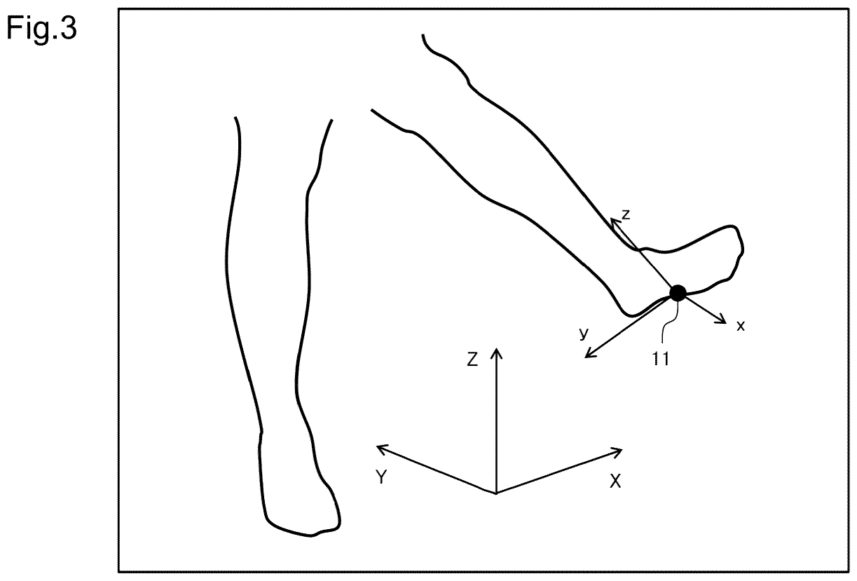

FIG. 3 is a conceptual diagram for explaining a local coordinate system (x-axis, y-axis, z-axis) set in the data acquisition device 11 and a world coordinate system (X-axis, Y-axis, Z-axis) set with respect to the ground in a case where the data acquisition device 11 is installed at a position corresponding to the back side of the arch of the foot. In the world coordinate system (X-axis, Y-axis, Z-axis), in a state where the user is standing upright, a lateral direction of the user is set to an X-axis direction (leftward direction is positive), a back face direction of the user is set to a Y-axis direction (rearward direction is positive), and a gravity direction is set to a Z-axis direction (vertically upward direction is positive). In the present example embodiment, a local coordinate system including the x direction, the y direction, and the z direction based on the data acquisition device 11 is set. In the present example embodiment, the same coordinate system is set for the left and right feet.

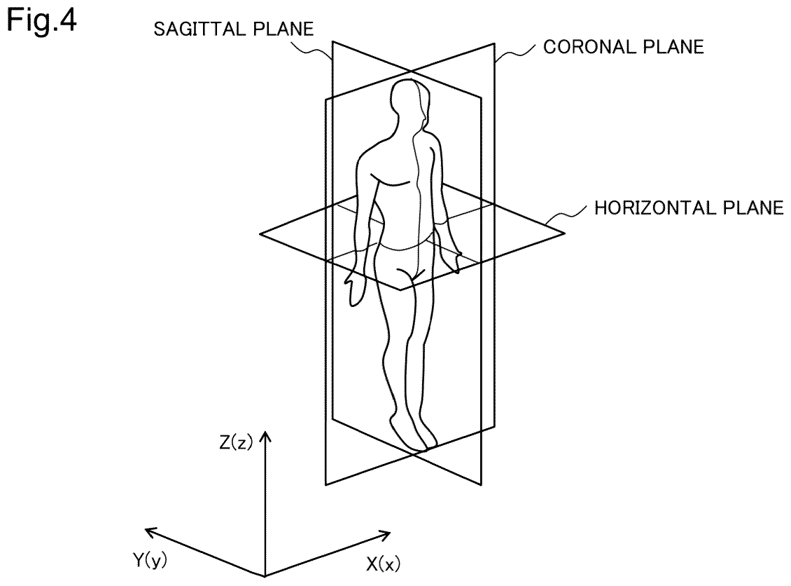

FIG. 4 is a conceptual diagram for explaining a face (also referred to as a human body surface) set for the human body. In the present example embodiment, a sagittal plane dividing the body into left and right, a coronal plane dividing the body into front and rear, and a horizontal plane dividing the body horizontally are defined. In the upright state as illustrated in FIG. 4, the world coordinate system coincides with the local coordinate system. In the present example embodiment, rotation in the sagittal plane with the x-axis as a rotation axis is defined as roll, rotation in the coronal plane with the y-axis as a rotation axis is defined as pitch, and rotation in the horizontal plane with the z-axis as a rotation axis is defined as yaw. A rotation angle in a sagittal plane with the x-axis as a rotation axis is defined as a roll angle, a rotation angle in a coronal plane with the y-axis as a rotation axis is defined as a pitch angle, and a rotation angle in a horizontal plane with the z-axis as a rotation axis is defined as a yaw angle. In the present example embodiment, when the body is viewed from the right side, clockwise rotation in the sagittal plane is defined as positive, and counterclockwise rotation in the sagittal plane is defined as negative.

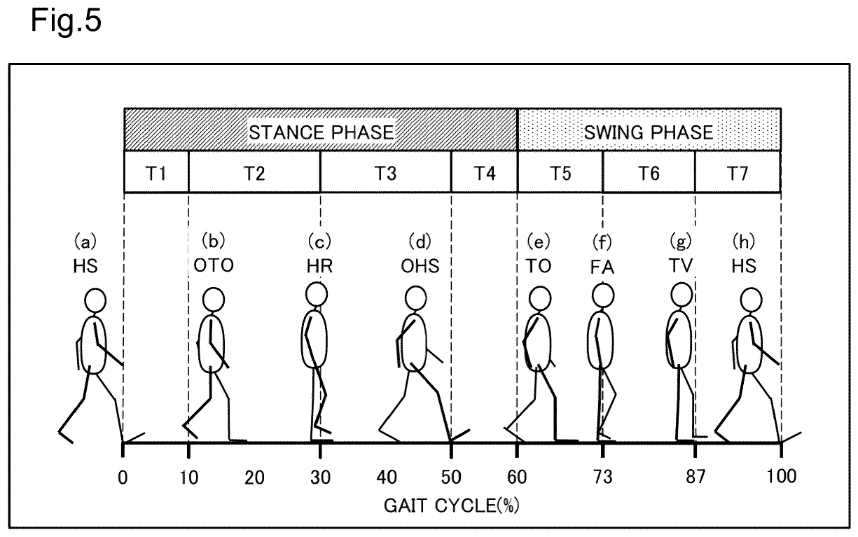

FIG. 5 is a conceptual diagram for explaining one gait cycle based on the left foot. One gait cycle based on the right foot is similar to that of the left foot. The horizontal axis of FIG. 5 is a normalized gait cycle with one gait cycle of the left foot as 100% with a time point at which the heel of the left foot lands on the ground as a starting point and a time point at which the heel of the left foot lands on the ground next as an ending point. The one gait cycle of one foot is roughly divided into a stance phase in which at least part of the back side of the foot is in contact with the ground and a swing phase in which the back side of the foot is away from the ground. The stance phase is further subdivided into an initial stance period T1, a mid-stance period T2 of standing, a terminal stance period T3 of standing, and a pre-swing period T4. The swing phase is further subdivided into an initial swing period T5, a mid-swing period T6, and a terminal swing period T7.

(a) of FIG. 5 illustrates an event (heel strike (HS)) in which the heel of the left foot is grounded. (b) of FIG. 5 illustrates an event (opposite toe off (OTO)) in which the toe of the right foot is away from the ground while the sole of the left foot is in contact with the ground. (c) of FIG. 5 illustrates an event (heel rise (HR)) in which the heel of the left foot is raised while the sole of the left foot is in contact with the ground. (d) of FIG. 5 illustrates an event (opposite heel strike (OHS)) in which the heel of the right foot is in contact with the ground. (e) of FIG. 5 llustrates an event (toe off (TO)) in which the toe of the left foot is away from the ground while the sole of the right foot is in contact with the ground. (f) of FIG. 5 illustrates an event (foot adjacent (FA)) in which the right foot and the left foot cross each other while the sole of the right foot is in contact with the ground. (g) of FIG. 5 illustrates an event (tibia vertical (TV)) in which the tibia of the left foot is substantially perpendicular to the ground while the sole of the right foot is in contact with the ground. (h) of FIG. 5 illustrates an event (heel strike (HS)) in which the heel of the left foot is grounded. (h) of FIG. 5 corresponds to the ending point of the gait cycle starting from (a) of FIG. 5 and corresponds to the starting point of the next gait cycle.

The detection device 12 acquires sensor data regarding the motion of the foot of the user. The detection device 12 generates a waveform (also referred to as a gait waveform) based on the time series data of the acquired sensor data. The detection device 12 detects a gait event from the generated gait waveform based on the condition set for each of the angle, the angular velocity, and the acceleration.

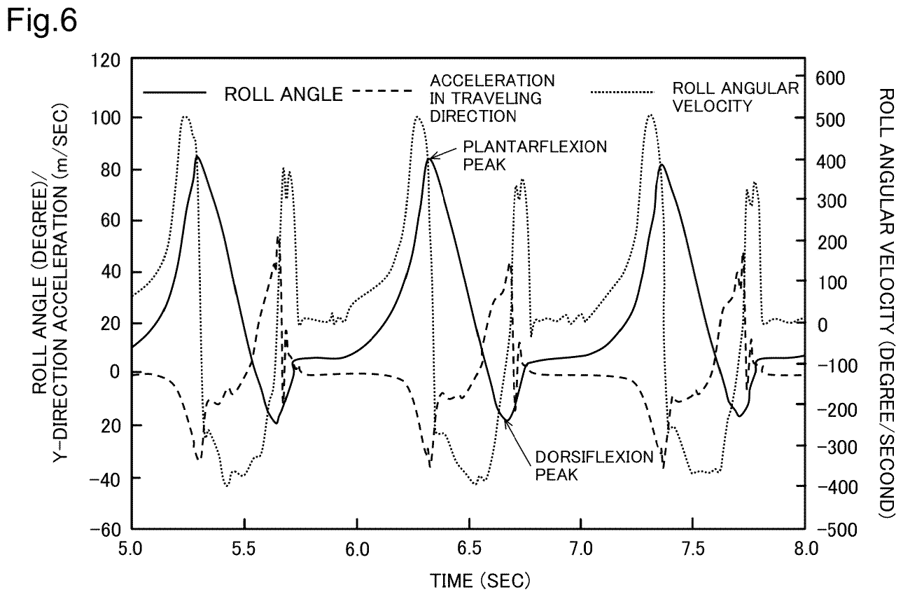

FIG. 6 is an example of a gait waveform of a person (also referred to as a healthy person) who has no abnormality in the left and right legs. FIG. 6 illustrates gait waveforms of the roll angle (solid line), the acceleration in the traveling direction (broken line), and the roll angular velocity (dotted line). The left axis represent the roll angle (solid line) and the acceleration in the traveling direction (broken line), and the right axis represents the roll angular velocity (dotted line). From the gait waveform of a healthy person, periodic peaks associated with a gait are clearly detected. For example, focusing on the gait waveform of the roll angle (solid line), a positive peak and a negative peak can be clearly distinguished. Focusing on the gait waveform of the roll angular velocity (dotted line), a positive peak appears in the vicinity of the peak of the gait waveform of the roll angle (solid line). The timing at which the peak appears in the gait waveform of each of the roll angle (solid line) and the roll angular velocity (dotted line) does not completely coincide with each other, but is associated with the timing at which plantarflexion of the foot is maximized and the timing at which dorsiflexion of the foot is maximized. Focusing on the gait waveform of the acceleration in the traveling direction (broken line), a characteristic change appears in the vicinity of the timing at which each of plantarflexion and dorsiflexion of the foot is maximized.

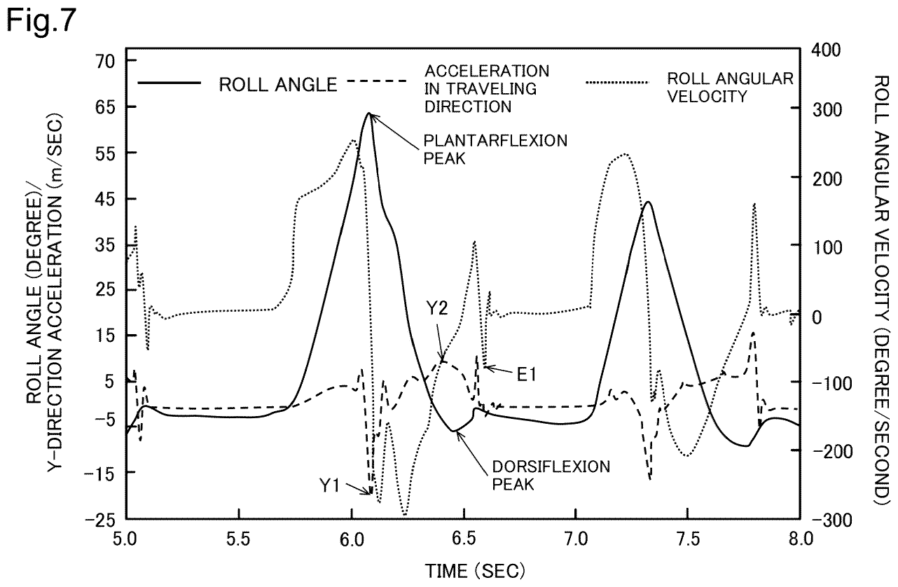

FIG. 7 is an example of a gait waveform related to the left foot of a person (hereinafter, referred to as a person with hemiplegia) who has paralysis on the left half of the body. The gait waveform and the axis in FIG. 7 are similar to those in the example of FIG. 6. Although the gait waveform of a person with hemiplegia includes a characteristic peak, a periodic peak associated with a gait is not clearly detected compared with that of the gait waveform of a healthy person. A common point of the gait waveforms between the person with hemiplegia and the healthy person is that a positive peak appears at the timing when plantarflexion of the foot is maximized in the gait waveform of the roll angle (solid line). Although not as clear as the gait waveform of a healthy person, a negative peak appears in the gait waveform of the roll angle of a person with hemiplegia at the timing when dorsiflexion of the foot is maximized. Even when the motion of the ankle is not successful, since there is rotation of the lower limb (hip joint) in order to move forward, a steep change appears in the gait waveform of the roll angular velocity of the person with hemiplegia in the vicinity of the timing of each of the toe off and the heel strike. When the timing at which plantarflexion of the foot is maximized arrives, the motion of the foot is accelerated forward when the foot is kicked forward, so that the absolute value of the acceleration in the traveling direction increases. At this time, since the forward direction is negative in the traveling direction (Y direction), the acceleration in the traveling direction shows a negative peak. On the other hand, when the timing at which the dorsiflexion of the foot is maximized arrives, the motion of the foot is rapidly decelerated, so that the absolute value of the acceleration in the traveling direction increases. At this time, since the forward direction is negative in the traveling direction (Y direction), the acceleration in the traveling direction shows a positive peak. In the present example embodiment, a gait event is detected based on characteristics commonly detected from the gait waveforms of a healthy person and a person with hemiparesis.

For example, the detection device 12 slides a window for a predetermined time in the time direction in the gait waveform, and detects the gait event based on the condition set for each of the angle, the angular velocity, and the acceleration. For example, the detection device 12 detects the gait event from the gait waveform based on an angle condition (also referred to as a first condition), an angular velocity condition (also referred to as a second condition), and an acceleration condition (also referred to as a third condition). The detection of the gait event by the detection device 12 will be described later.

### [Data Acquisition Device]

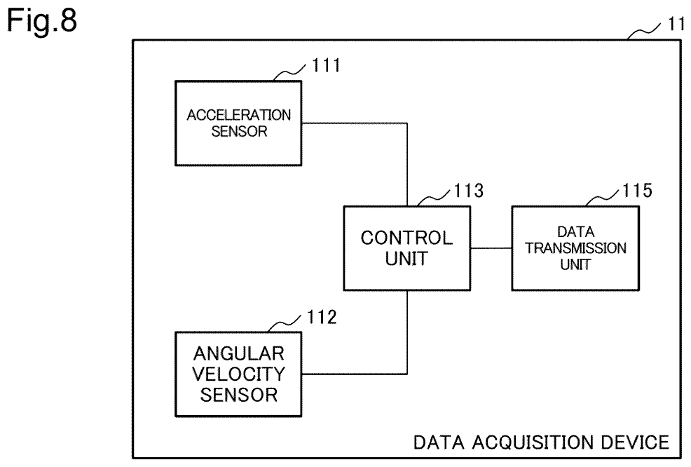

Next, details of the data acquisition device 11 will be described with reference to the drawings. FIG. 8 is a block diagram illustrating an example of a detailed configuration of the data acquisition device 11. The data acquisition device 11 includes an acceleration sensor 111, an angular velocity sensor 112, a control unit 113, and a data transmission unit 115. The data acquisition device 11 includes a power supply (not illustrated). In the following description, each of the acceleration sensor 111, the angular velocity sensor 112, the control unit 113, and the data transmission unit 115 will be described as an operation subject, but the data acquisition device 11 may be regarded as an operation subject.

The acceleration sensor 111 is a sensor that measures accelerations (also referred to as spatial accelerations) in three axial directions. The acceleration sensor 111 outputs the measured acceleration to the control unit 113. For example, a sensor of a piezoelectric type, a piezoresistive type, a capacitance type, or the like can be used as the acceleration sensor 111. As long as the sensor used for the acceleration sensor 111 can measure an acceleration, the measurement method is not limited.

The angular velocity sensor 112 is a sensor that measures angular velocities in three axial directions (also referred to as spatial angular velocities). Angular velocity sensor 112 outputs the measured angular velocity to control unit 113. For example, a sensor of a vibration type, a capacitance type, or the like can be used as the angular velocity sensor 112. As long as the sensor used for the angular velocity sensor 112 can measure an angular velocity, the measurement method is not limited.

The control unit 113 acquires the accelerations and the angular velocities in three axial directions from the acceleration sensor 111 and the angular velocity sensor 112, respectively. The control unit 113 converts the acquired acceleration and angular velocity into digital data to output the converted digital data (also referred to as sensor data) to the data transmission unit 115. The sensor data includes at least acceleration data (including acceleration vectors in three axial directions) obtained by converting acceleration of analog data into digital data and angular velocity data (including angular velocity vectors in three axial directions) obtained by converting angular velocity of analog data into digital data. The acquisition times of the acceleration data and the angular velocity data are associated with the acceleration data and the angular velocity data. The control unit 113 may be configured to output sensor data obtained by adding correction such as a mounting error, temperature correction, and linearity correction to the acquired acceleration data and angular velocity data. The control unit 113 may generate angle data in three axial directions using the acquired acceleration data and angular velocity data.

For example, the control unit 113 is a microcomputer or a microcontroller that performs overall control and data processing of the data acquisition device 11. For example, the control unit 113 includes a central processing unit (CPU), a random access memory (RAM), a read only memory (ROM), a flash memory, and the like. The control unit 113 controls the acceleration sensor 111 and the angular velocity sensor 112 to measure the angular velocity and the acceleration. For example, the control unit 113 performs analog-to-digital conversion (AD conversion) on physical quantities (analog data) such as the measured angular velocity and acceleration, and stores the converted digital data in the flash memory. The physical quantity (analog data) measured by each of the acceleration sensor 111 and the angular velocity sensor 112 may be converted into digital data in each of the acceleration sensor 111 and the angular velocity sensor 112. The digital data stored in the flash memory is output to the data transmission unit 115 at a predetermined timing.

The data transmission unit 115 acquires sensor data from the control unit 113. The data transmission unit 115 transmits the acquired sensor data to the detection device 12. The data transmission unit 115 may transmit the sensor data to the detection device 12 via a wire such as a cable, or may transmit the sensor data to the detection device 12 via wireless communication. For example, the data transmission unit 115 is configured to transmit sensor data to the detection device 12 via a wireless communication function (not illustrated) conforming to a standard such as Bluetooth (registered trademark) or WiFi (registered trademark). The communication function of the data transmission unit 115 may conform to a standard other than Bluetooth (registered trademark) or WiFi (registered trademark).

### [Detection Device]

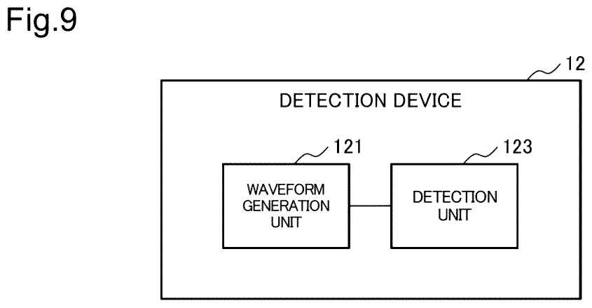

Next, details of the detection device 12 will be described with reference to the drawings. FIG. 9 is a block diagram illustrating an example of a configuration of the detection device 12. The detection device 12 includes a waveform generation unit 121 and a detection unit 123.

The waveform generation unit 121 acquires sensor data from the data acquisition device 11 (sensor) installed on the footwear worn by the pedestrian. Using the sensor data, the waveform generation unit 121 generates time series data (also referred to as a gait waveform) associated with a gait of the pedestrian wearing the footwear on which the data acquisition device 11 is installed.

For example, the waveform generation unit 121 generates time series data such as a spatial acceleration and a spatial angular velocity. The waveform generation unit 121 integrates the spatial acceleration and the spatial angular velocity to generate time series data such as the spatial velocity, the spatial angle (foot sole angle), and the spatial locus. The waveform generation unit 121 generates time series data at a predetermined timing or time intervals set in accordance with a general gait cycle or a gait cycle unique to the user. The timing at which the waveform generation unit 121 generates the time series data can be set in any manner. For example, the waveform generation unit 121 is configured to continue to generate time series data during a period in which a gait of the user is continued. The waveform generation unit 121 may be configured to generate time series data at a specific time.

The detection unit 123 detects a gait event from the gait waveform generated by the waveform generation unit 121 based on the condition set for each of the angle, the angular velocity, and the acceleration. For example, the detection unit 123 slides a window for a predetermined time in the gait waveform in the time direction, and detects the gait event based on the condition set for each of the angle, the angular velocity, and the acceleration. For example, the detection unit 123 detects a gait event from the gait waveform generated by the waveform generation unit 121 based on an angle condition (also referred to as a first condition), an angular velocity condition (also referred to as a second condition), and an acceleration condition (also referred to as a third condition). In the present example embodiment, an example of detecting a gait event based on the condition set for each of an angle (roll angle) in the sagittal plane, an angular velocity (roll angular velocity) in the sagittal plane, and an acceleration (acceleration in the traveling direction) in the sagittal plane (traveling direction) will be described.

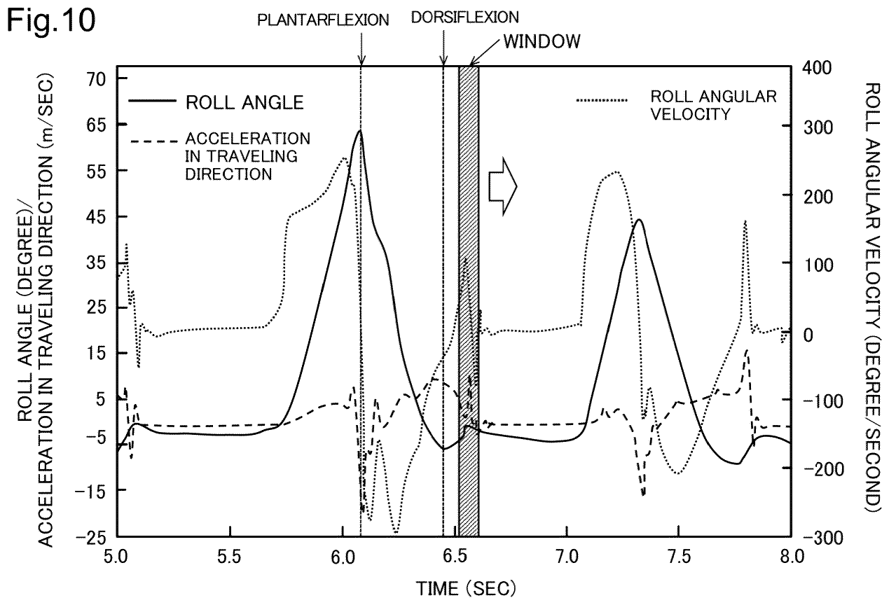

FIG. 10 is a conceptual diagram for explaining an example in which a window for a predetermined time is slid in the time direction in the gait waveform of FIG. 7 and a gait event is detected based on the condition set for each of the angle, the angular velocity, and the acceleration in the sagittal plane. The detection unit 123 slides a window for a predetermined time in the time direction with respect to the gait waveform related to each of the angle, the angular velocity, and the acceleration in the sagittal plane, and detects the gait event based on the condition set for each of the angle, the angular velocity, and the acceleration.

The time width of the window for the predetermined time is set to a width in which the gait event can be detected from the gait waveform regarding each of the angle, the angular velocity, and the acceleration in the sagittal plane. For example, in a case where the measurement data of the gait waveform is measured at 100 points (100 hertz) per second, the window is set to a time width with 3 to 7 points. When the time width is excessively increased, there is a high possibility that an inflection point included in the gait waveform is erroneously detected. Therefore, the time width of the window is preferably set to about seven points. In a case where the measurement interval of the measurement data of the gait waveform is not 100 hertz, the time width of the window may be set in accordance with each measurement interval.

An example of detection of the plantarflexion peak and the dorsiflexion peak will be described using a specific example. Hereinafter, an example will be described in which a peak is detected based on the first condition, and which of the plantarflexion or the dorsiflexion the detected peak is associated with is determined using the second condition and the third condition.

<First Condition>

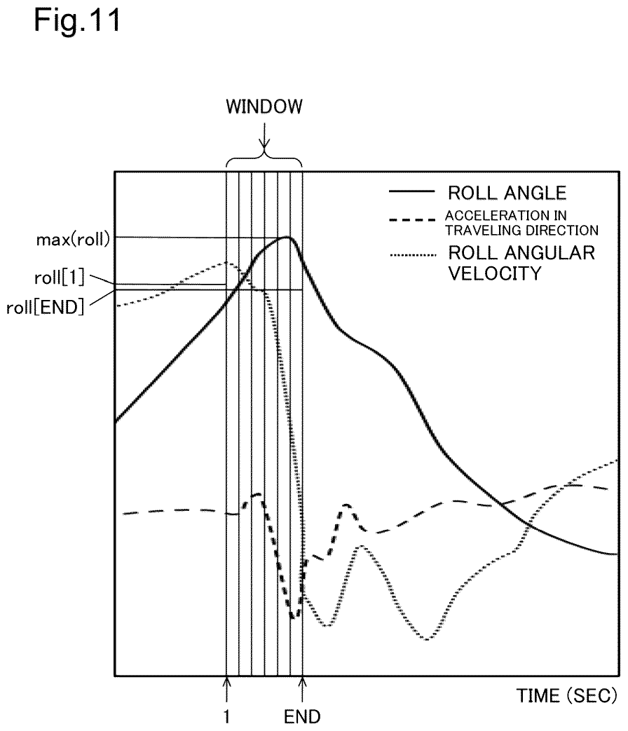

The first condition is a condition for detecting a peak from a gait waveform represented by an angle (roll angle) in rotation in the sagittal plane. FIG. 11 is a conceptual diagram for explaining an example of detecting a peak from a gait waveform represented by a roll angle based on the first condition. For example, the detection unit 123 slides a window for a predetermined time in the time direction in the gait waveform represented by the roll angle, and detects the peak based on the first condition.

In FIG. 11, the window is divided into six regions by seven lines (also referred to as measurement lines) including both right and left ends in the time direction. An identification number (ID) is assigned to each timing related to each of the seven measurement lines in order from the left. In the present example embodiment, IDs 1, 2, 3, 4, 5, 6, and END are assigned to the seven measurement lines in order from the left. The value of the roll angle at ID(1) of the timing (also referred to as a start point) of the leftmost measurement line (left end) inside the window is described as roll[1]. The value of the roll angle at ID(END) of the timing (also referred to as an ending point) of the rightmost measurement line (right end) inside the window is described as roll[END]. The value of the largest roll angle inside the window is described as max(roll). The smallest roll angle value inside the window is described as min(roll).

For example, the first condition includes a condition (also referred to as a first detection condition) for detecting an upward convex peak inside the window from the gait waveform represented by the roll angle, and a condition (also referred to as a first determination condition) for determining that the detected peak is not noise.

The first detection condition is a condition that the roll angle at the start point is smaller than the maximum value of the roll angle inside the window, and the roll angle at the ending point is smaller than the maximum value of the roll angle inside the window. When the following Equations 1 and 2 are both satisfied, the first detection condition is satisfied.

max(roll)>roll[1] (1)

max(roll)>roll[END] (2)

When a downward convex peak is detected inside the window, the condition that min(roll) is smaller than roll[1] and roll[END] may be satisfied.

The first determination condition is a condition that a value obtained by subtracting the values of the roll angles at the start point (ID=1) and the ending point (ID=END) from the maximum value of the roll angle inside the window exceeds a first threshold value Th₁. The first threshold value Th₁ is set according to the magnitude of noise included in the gait waveform represented by the roll angle. For example, the first threshold value Th₁ is set to 0.2 degrees. When the following Equation 3 is satisfied, the first determination condition is satisfied, and a peak is detected from the gait waveform represented by the roll angle.

max(roll)-min(roll(1),roll(END))>T₁ (3)

When a downward convex peak is detected inside the window, the condition that min (roll) is smaller than the smaller one of roll[1] and roll[END] may be satisfied.

<Second Condition>

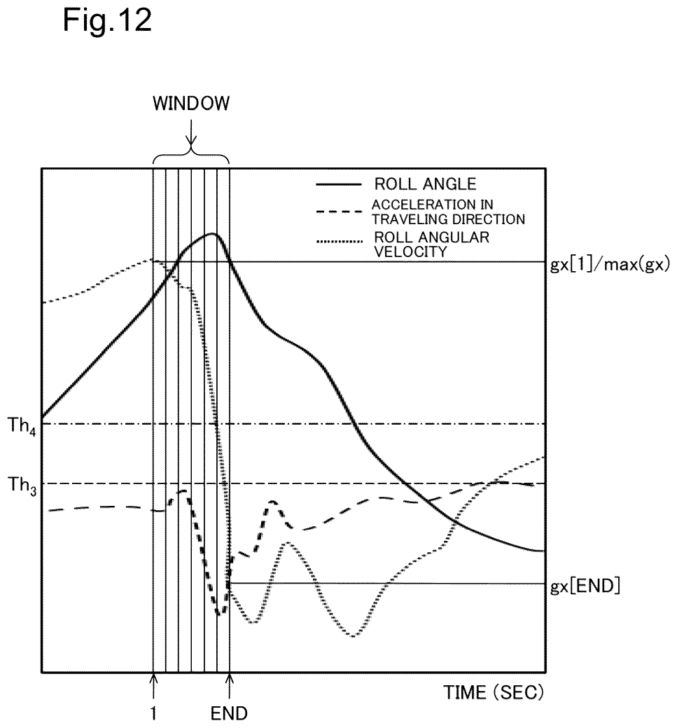

The second condition is a condition for determining whether the peak detected under the first condition corresponds to either the plantarflexion or the dorsiflexion using the gait waveform represented by the angular velocity (roll angular velocity) in the rotation in the sagittal plane. FIG. 12 is a conceptual diagram for explaining an example of determining whether a peak associated with the maximum of the plantarflexion or the dorsiflexion of the foot is included in the gait waveform represented by the roll angular velocity inside the window based on the second condition. For example, the detection unit 123 determines, based on the second condition, whether the peak detected under the first condition corresponds to either the plantarflexion or the dorsiflexion in the gait waveform represented by the roll angular velocity inside the window for a predetermined time.

In FIG. 12, the value of the roll angular velocity at the timing (start point: ID=1) of the leftmost measurement line (left end) inside the window is described as gx[1]. The value of the roll angular velocity at the timing (ending point: ID=END) of the rightmost measurement line (right end) inside the window is described as gx[END]. The value of the largest roll angular velocity inside the window is described as max(gx). The smallest roll angular velocity value inside the window is described as min(gx).

For example, the second condition includes a second detection condition and a second determination condition. The second detection condition is a condition for detecting a location where an amount of change in the roll angular velocity is steep inside the window. The second determination condition is a condition for determining which maximum of the plantarflexion and the dorsiflexion of the foot a location where the detected amount of change is large is associated with.

The second detection condition is a condition that a value obtained by subtracting the roll angular velocity at either the start point or the ending point from the maximum value of the roll angular velocity inside the window is larger than a second threshold value Th₂. For example, the second threshold value Th₂ is set to 50 degrees/second. In a case where any one of the following Equations 4 and 5 is satisfied, the second detection condition is satisfied.

max(gx)-gx[1]>T₂ (4)

max(gx)-gx[END]>T₂ (5)

When Equation 4 is satisfied, it is estimated that the peak detected under the first condition corresponds to the plantarflexion. On the other hand, when Equation 5 is satisfied, it is estimated that the peak detected under the first condition corresponds to the dorsiflexion.

The second determination condition is a condition that the value of the roll angular velocity at the start point (ID=1) is smaller than a third threshold value Th₃ or the value of the roll angular velocity at the ending point (ID=END) is a value between the third threshold value Th₃ and a fourth threshold value Th₄. For example, the third threshold value Th₃ is set to −70 degrees/second. For example, the fourth threshold value Th₄ is set to 15 degrees/second. When the following Equation 6 or 7 is satisfied, the second determination condition is satisfied.

gx[1]<T₃ (6)

T₃<gx[END]<T₄ (7)

When Equation 6 is satisfied, it is determined that the peak detected under the first condition corresponds to the plantarflexion. On the other hand, when Equation 7 is satisfied, it is determined that the peak detected under the first condition corresponds to the dorsiflexion.

The detection unit 123 detects a peak that satisfies both the second detection condition and the second determination condition as a peak associated with the maximum of the plantarflexion or the dorsiflexion of the foot. In the case of the example of FIG. 12, since the above Equation (6) is satisfied, the detection unit 123 determines that the peak detected based on the second detection condition is a peak associated with the maximum of the plantarflexion of the foot. In a case where the above Equation (7) is satisfied, the detection unit 123 determines that the peak detected based on the second detection condition is a peak associated with the maximum of the dorsiflexion of the foot.

<Third Condition>

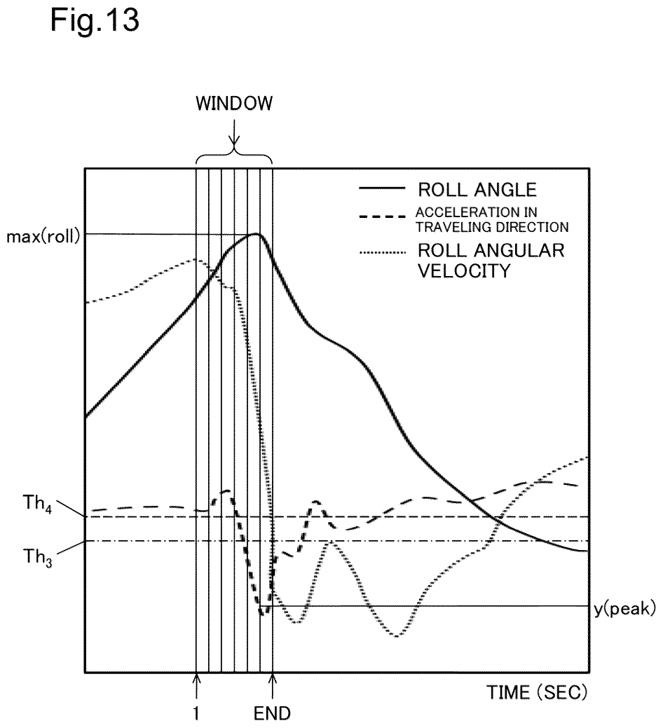

The third condition is a condition for determining whether the peak detected under the first condition is associated with the maximum of the plantarflexion or the dorsiflexion of the foot using the gait waveform represented by the acceleration (acceleration in the traveling direction) in the sagittal plane (traveling direction). FIG. 13 is a conceptual diagram for explaining an example of detecting a gait event based on the third condition from the gait waveform represented by the acceleration in the traveling direction. For example, the detection unit 123 determines, based on the third condition, whether the peak detected under the first condition corresponds to either the plantarflexion or the dorsiflexion in the gait waveform represented by the acceleration in the traveling direction inside the window for a predetermined time. The determination based on the third condition may be performed together with the determination based on the second determination condition of the second condition, or may be performed instead of the second determination condition of the second condition. When the determination based on the second condition is sufficient, the determination based on the third condition may not be performed.

In FIG. 13, the value of the acceleration in the traveling direction at the timing (start: ID=1) of the leftmost measurement line (left end) inside the window is described as y[1]. The value of the acceleration in the traveling direction at the timing (ending point: ID=END) of the rightmost measurement line (right end) inside the window is described as y[END]. The peaks in the acceleration in the traveling direction detected inside the window may include an upward convex peak and a downward convex peak. The largest value of the acceleration in the traveling direction inside the window is described as max(y). The smallest value of the acceleration in the traveling direction inside the window is described as min(y). The value of the acceleration in the traveling direction related to the ID of the peak of the roll angle detected inside the window based on the first condition is described as y(peak).

The third condition includes a condition (also referred to as a third determination condition) for determining which of the plantarflexion peak or the dorsiflexion peak the peak detected inside the window is. The third determination condition is a condition that the value of y(peak) is smaller than a fifth threshold value Th₅ or the value of y(peak) is larger than a sixth threshold value Th₆. For example, the fifth threshold value Th₅ is set to −0.4 g (g is gravitational acceleration). For example, the sixth threshold value Th₆ is set to +0.2 g. When the following Equation 8 or 9 is satisfied, the third determination condition is satisfied.

y(peak)<T₅ (8)

y(peak)>T₆ (9).

The detection unit 123 detects a peak that satisfies the third determination condition as a peak associated with the maximum of the plantarflexion or the dorsiflexion of the foot. The detection unit 123 determines that the peak satisfying the above Equation (8) is a peak associated with the maximum of the plantarflexion of the foot. The detection unit 123 determines that the peak satisfying the above Equation (9) is a peak associated with the maximum of the dorsiflexion of the foot.

Based on the first determination condition, the second determination condition, and the third determination condition, the detection unit 123 detects a gait event accompanying a gait of the user. For example, the detection unit 123 detects the timing of the peak associated with the maximum of the plantarflexion of the foot as the timing of the toe off. For example, the detection unit 123 detects the timing of the peak associated with the maximum of the dorsiflexion of the foot as the timing of the heel strike. For example, the detection unit 123 detects various gait events from the gait waveform based on the toe off or the heel strike. For example, the detection unit 123 detects various gait events from the gait waveform based on the feature detected from the gait waveform with reference to the toe off or the heel strike. For example, the detection unit 123 detects various gait events from the gait waveform based on the lapse of time or time allocation based on the toe off or the heel strike. For example, the detection unit 123 detects a gait event such as an opposite toe off, a heel rise, an opposite heel strike foot adjacent, and a tibia vertical with the toe off or the heel strike as a reference. For example, the result of detection by the detection unit 123 can be used for verification of the locus of a gait, the gait speed, the stride length, the symmetry of gait, the length of the gait phase, and the like.

(Operation)

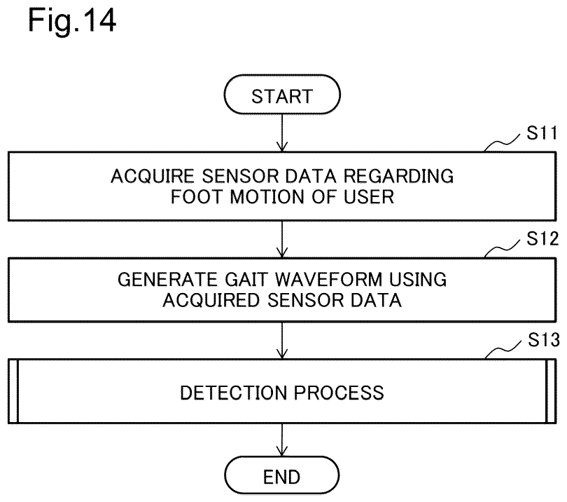

Next, the operation of the detection device 12 of the detection system 1 of the present example embodiment will be described with reference to the drawings. FIG. 14 is a flowchart for explaining an outline of the operation of the detection device 12. Details of the operation of the detection device 12 are as described regarding the above-described configuration. In the description along the flowchart of FIG. 14, the detection device 12 will be described as an operation subject. In FIG. 14, first, the detection device 12 acquires sensor data regarding the motion of the foot (step S11).

Next, the detection device 12 generates time series data (also referred to as a gait waveform) using the acquired sensor data (step S12).

Next, the detection device 12 performs detection process on the generated gait waveform (step S13). For example, the detection device 12 detects a peak satisfying the first condition, the second condition, and the third condition from the gait waveforms represented by the roll angle, the roll angular velocity, and the acceleration in the traveling direction, and determines a gait event related to the detected peak. [Detection Processing]

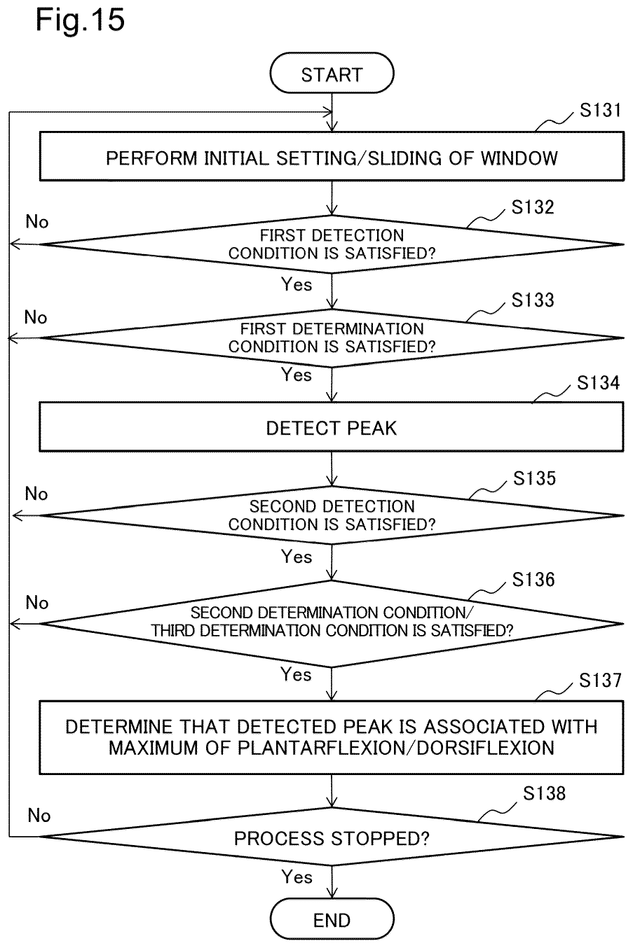

Next, detection processing by the detection device 12 will be described with reference to the drawings. FIG. 15 is a flowchart for explaining detection processing by the detection device 12. In the description along the flowchart of FIG. 15, the detection device 12 will be described as an operation subject.

First, the detection device 12 sets a window at an initial position of the gait waveform having the roll angle, the roll angular velocity, and the acceleration in the traveling direction as a set (step S131). Setting the window at the initial position of the gait waveform is also referred to as initial setting. For example, the initial position is a position where the measurement line of the start point (ID=1) of the window overlaps at the earliest time in the gait waveform.

Next, when the first detection condition is satisfied with respect to the gait waveform represented by the roll angle (Yes in step S132), a timing at which the roll angle indicates a maximum (or minimum) value is detected as a peak candidate. On the other hand, when the first detection condition is not satisfied (No in step S132), the process returns to step S131, and the detection device 12 slides the window. For example, the detection device 12 slides the window to a position where the measurement line at the start point (ID=1) of the window overlaps the measurement line at the ending point (ID=END).

Next to step S132, when the first determination condition is satisfied (Yes in step S133), the detection device 12 detects the candidate for the peak detected in step S132 as a peak (step S134). On the other hand, when the first determination condition is not satisfied (No in step S133), the process returns to step S131, and the detection device 12 slides the window.

After step S134, the detection unit 123 verifies whether the roll angular velocity satisfies the second detection condition inside the window in which the peak is detected (step S136). When the roll angular velocity satisfies the second detection condition (Yes in step S135), the detection unit 123 determines which maximum of the plantarflexion and the dorsiflexion of the foot the peak is associated with based on the second determination condition or the third determination condition (step S136). On the other hand, when the roll angular velocity does not satisfy the second detection condition (No in step S135), the process returns to step S131, and the detection device 12 slides the window.

Next to step S135, when the roll angular velocity satisfies the second determination condition or the acceleration in the traveling direction satisfies the third determination condition (Yes in step S136), the detection unit 123 determines that the peak is associated with the maximum of either the plantarflexion or the dorsiflexion (step S137). For example, in a case where the roll angular velocity is smaller than the third threshold value, the detection unit 123 determines that the detected peak corresponds to the plantarflexion. For example, when the roll angular velocity is a value between the third threshold value and the fourth threshold value, the detection unit 123 determines that the detected peak is associated with the maximum of the dorsiflexion of the foot. For example, when the acceleration in the traveling direction at the timing of the peak is smaller than the fifth threshold value, the detection unit 123 determines that the detected peak is associated with the maximum of the plantarflexion of the foot. For example, when the acceleration in the traveling direction at the timing of the peak is larger than the sixth threshold value, it is determined that the detected peak is associated with the maximum of the dorsiflexion of the foot. In step S136, when determining which maximum of the plantarflexion and the dorsiflexion of the foot the timing of the detected peak is associated with, the detection unit 123 may use both the roll angular velocity and the acceleration in the traveling direction, or may use one of the roll angular velocity and the acceleration in the traveling direction.

After step S137, when the process is not stopped (No in step S138), the process returns to step S131. On the other hand, when the process is stopped (Yes in step S138), the process along the flowchart of FIG. 15 is ended.

As described above, the detection system of the present example embodiment includes the data acquisition device and the detection device.

The data acquisition device measures the spatial acceleration and the spatial angular velocity, generates sensor data based on the measured spatial acceleration and spatial angular velocity to transmit the generated sensor data to the estimation device. The detection device includes the waveform generation unit and the detection unit. The waveform generation unit generates a gait waveform using sensor data related to the motion of the foot. The detection unit detects the gait event from the gait waveform based on the condition set for each of the angle, the angular velocity, and the acceleration in the sagittal plane.

In the present example embodiment, the gait event is detected from the gait waveform based on the condition set for each of the angle, the angular velocity, and the acceleration in the sagittal plane. Therefore, according to the present example embodiment, a gait event can be detected based on the gait waveform not only for a gait of a healthy person but also for a gait of a person with physical disabilities.

In an aspect of the present example embodiment, the detection unit sets a window for a predetermined time in the gait waveform represented by each of the angle, the angular velocity, and the acceleration in the sagittal plane, and detects the gait event by sliding the window in the time direction. According to the present aspect, since the gait waveform is verified in the local region inside the window, it is possible to detect a feature that is difficult to grasp from the entire gait waveform. Therefore, according to the present aspect, even when the gait waveform includes many inflection points, the gait event can be detected based on the gait waveform.

In an aspect of the present example embodiment, the detection unit detects a peak from a gait waveform represented by an angle in the sagittal plane based on a first condition including a first detection condition and a first determination condition. The first detection condition is a condition for detecting a peak candidate based on a magnitude relationship between a value of an angle at timing of both ends of the window and a maximum angle inside the window in the gait waveform represented by an angle in the sagittal plane. The first determination condition is a condition for determining whether a peak candidate is a peak. According to the present aspect, it is possible to detect a peak from which noise has been removed from the gait waveform represented by an angle in the sagittal plane.

In an aspect of the present example embodiment, the detection unit determines which maximum of the plantarflexion and the dorsiflexion of the foot the peak detected from the gait waveform represented by the angle in the sagittal plane is associated with based on the second condition including the second detection condition and the second determination condition. The second detection condition is a condition for detecting a location where the amount of change in the angular velocity inside the window is steep in the gait waveform represented by the angular velocity in the sagittal plane. The second determination condition is a condition for determining which of the plantarflexion peak and the dorsiflexion peak a location where the amount of change in the to angular velocity inside the window is steep is associated with based on the magnitude relationship between the value of the angular velocity at the timing of both ends of the window and the maximum angular velocity inside the window. According to the present aspect, by combining the second detection condition and the second determination condition, it is possible to determine which maximum of the plantarflexion and the dorsiflexion of the foot the peak detected from the gait waveform represented by an angle in the sagittal plane is associated with.

In an aspect of the present example embodiment, the detection unit determines which maximum of the plantarflexion and the dorsiflexion of the foot the peak detected from the gait waveform represented by the angle in the sagittal plane is associated with based on the second detection condition and the third determination condition (third condition). The second detection condition is a condition for detecting a location where the amount of change in the angular velocity inside the window is steep in the gait waveform represented by the angular velocity in the sagittal plane. The third determination condition is a condition for determining which of the plantarflexion peak and the dorsiflexion peak the peak is associated with based on the value of the acceleration in the traveling direction in the sagittal plane at the timing of the peak detected inside the window. According to the present aspect, by combining the second detection condition and the third determination condition, it is possible to determine which maximum of the plantarflexion and the dorsiflexion of the foot the peak detected from the gait waveform represented by an angle in the sagittal plane is associated with.

The method of the present example embodiment can be applied not only to a gait of a person with hemiplegia but also to a gait of a person with physical disability due to Parkinson's disease, rheumatism, knee osteoarthritis, osteoporosis, pronation/supination, hallux valgus, or the like. The method of the present example embodiment can be applied to a gait of a person who has an artificial joint in one foot or a person who has injured one foot. For example, when the transition of the gait waveform is verified, the method of the present example embodiment can also be used for monitoring the recovery state of a leg injury or the like.

### Second Example Embodiment

Next, a detection device according to a second example embodiment will be described with reference to the drawings. The detection device of the present example embodiment is different from the detection device of the first example embodiment in that the gait state is determined using the detection result of plantarflexion/dorsiflexion of the foot. In the present example embodiment, an example will be described in which the gait state is determined by distinguishing a period (stance phase) in which the foot is in contact with the ground from a period (swing phase) in which the foot is away from the ground. Hereinafter, detailed description of parts similar to those of the first example embodiment will be omitted.

(Configuration)

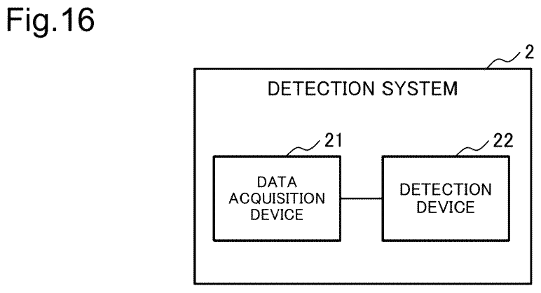

FIG. 16 is a block diagram illustrating a configuration of a detection system 2 of the present example embodiment. The detection system 2 includes a data acquisition device 21 and a detection device 22. The data acquisition device 21 and the detection device 22 may be connected by wire or wirelessly. The data acquisition device 21 and the detection device 22 may be configured by a single device. The detection system 2 may be configured only by the detection device 22 excluding the data acquisition device 21 from the configuration of the detection system 2. Although only one data acquisition device 21 is illustrated in FIG. 16, (two in total) data acquisition devices 21 may be disposed one by one in association with the left and right feet. The data acquisition device 21 has a configuration similar to that of the data acquisition device 11 of the first example embodiment. Hereinafter, the detection device 22 different from that of the first example embodiment will be described focusing on differences from the first example embodiment.

### [Detection Device]

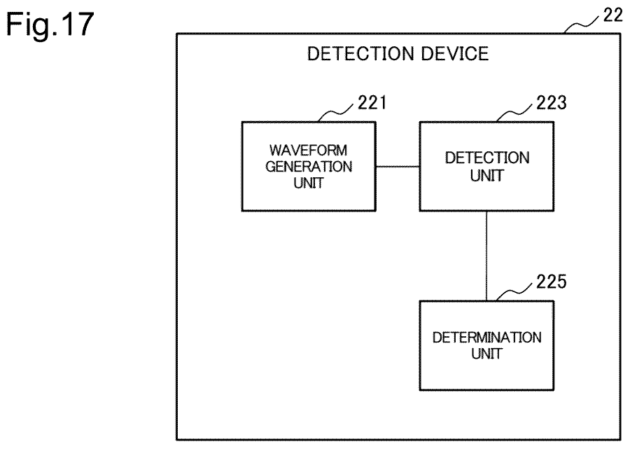

FIG. 17 is a block diagram illustrating an example of a configuration of the detection device 22 of the present example embodiment. The detection device 22 includes a waveform generation unit 221, a detection unit 223, and a determination unit 225. Since the waveform generation unit 221 and the detection unit 223 has a configuration similar to the related configuration of the detection device 12 of the first example embodiment, a detailed description thereof will be omitted.

The determination unit 225 acquires a result of detection by the detection unit 223. For example, the determination unit 225 acquires a detection result indicating which maximum of the plantarflexion and the dorsiflexion of the foot the peak satisfying the first condition is associated with. In the following description, a peak associated with the maximum of the plantarflexion of the foot is referred to as a plantarflexion peak, and a peak associated with the maximum of the dorsiflexion of the foot is referred to as a dorsiflexion peak.

The determination unit 225 determines the gait state based on the acquired detection result. For example, determination unit 225 determines a period between the plantarflexion peaks that are consecutive as one step. For example, determination unit 225 determines a period between the dorsiflexion peaks that are consecutive as one step. For example, the determination unit 225 determines a period between the plantarflexion peak and the dorsiflexion peak that are consecutive as a swing phase. For example, the determination unit 225 detects a section between the dorsiflexion peak and the plantarflexion peak that are consecutive as a stance phase. For example, the determination unit 225 outputs information in which the gait waveform generated by the waveform generation unit 221 is associated with the determination results regarding the swing phase and the stance phase. For example, the information output from the determination unit 225 is displayed on a screen of a display device (not illustrated).

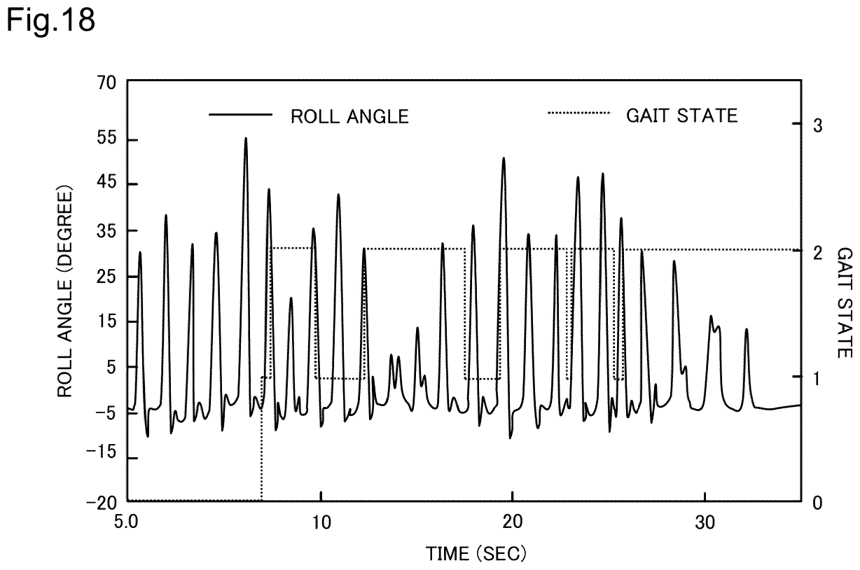

FIG. 18 is a conceptual diagram for explaining an example of a gait state determined using the related art. FIG. 18 is an example of a graph in which the determination result of the gait state based only on the gait waveform represented by the roll angle is superimposed on the gait waveform represented by the roll angle of the person with hemiplegia. In the graph of FIG. 18, the numerical values indicating the gait state are 0 in the initial state, 1 in the stance phase, and 2 in the swing phase. The gait waveform represented by the roll angle of a person with hemiplegia has a clear plantarflexion peak, but includes a complicated inflection point, and thus it is difficult to determine the dorsiflexion peak. That is, it is difficult to accurately determine the gait state of the person with hemiplegia only from the gait waveform represented by the roll angle.

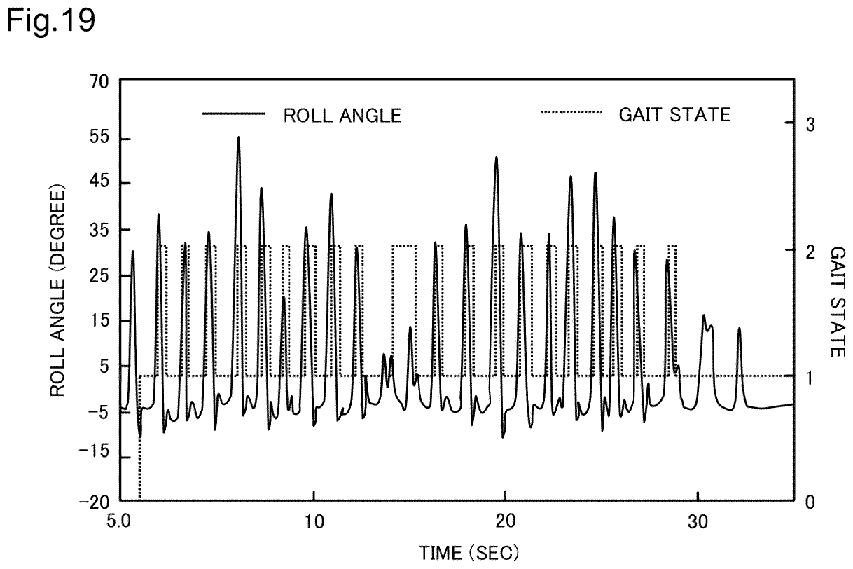

FIG. 19 is a conceptual diagram for explaining an example of the gait state determined by the determination unit 225. FIG. 19 is an example of a graph in which the determination result of the gait state by the determination unit 225 is superimposed on the gait waveform represented by the roll angle of the person with hemiplegia. In the graph of FIG. 19, the numerical values indicating the gait state are 0 in the initial state, 1 in the stance phase, and 2 in the swing phase. As illustrated in FIG. 19, according to the method of the present example embodiment, even in the gait waveform of the person with hemiplegia, a period from the plantarflexion peak to the dorsiflexion peak can be determined as the swing phase, and a period from the dorsiflexion peak to the plantarflexion peak can be determined as the stance phase. Therefore, according to the method of the present example embodiment, the gait state of the person with hemiplegia can be accurately determined. That is, according to the present example embodiment, since the timing corresponding to plantarflexion/dorsiflexion of the foot can be accurately detected from the gait waveform represented by each of the roll angle, the roll angular velocity, and the acceleration in the traveling direction based on the first condition, the second condition, and the third condition, the gait state of the person with hemiplegia can be accurately determined. According to the method of the present example embodiment, regarding the gait waveform of the healthy person, the timing corresponding to plantarflexion/dorsiflexion of the foot can be accurately detected, so that the gait state of the person with hemiplegia can be accurately determined. For example, the number of steps is counted by the method of the present example embodiment, and when the number of steps of three or more steps is detected, it can be detected that a stable gait has started. As described above, by using the method of the present example embodiment, even for a person having an abnormality in gait due to the influence of hemiplegia or the like, the gait state can be verified as in a healthy person.

As described above, the detection system of the present example embodiment includes the data acquisition device and the detection device. The data acquisition device measures the spatial acceleration and the spatial angular velocity, generates sensor data based on the measured spatial acceleration and spatial angular velocity to transmit the generated sensor data to the estimation device. The detection device includes a waveform generation unit, a detection unit, and a determination unit. The waveform generation unit generates a gait waveform using sensor data related to the motion of the foot. The detection unit detects the gait event from the gait waveform based on the condition set for each of the angle, the angular velocity, and the acceleration in the sagittal plane. The determination unit determines the gait state based on the peak detected by the detection unit.

According to the present example embodiment, by determining the gait state based on the peak detected by the detection unit, the gait event can be detected based on the gait waveform not only for a gait of a healthy person but also for a gait of a person with physical disability.

In an aspect of the present example embodiment, the determination unit determines a period between the plantarflexion peak and the dorsiflexion peak that are consecutive as a swing phase, and determines a section between the dorsiflexion peak and the plantarflexion peak that are consecutive as a stance phase. The determination unit outputs information indicating which of the swing phase and the stance phase the time of the gait waveform generated by the waveform generation unit is associated with. According to the present aspect, by associating the determination result of the determination unit with the gait waveform, it is possible to verify what kind of gait state the feature included in the gait waveform is caused by.

### Third Example Embodiment

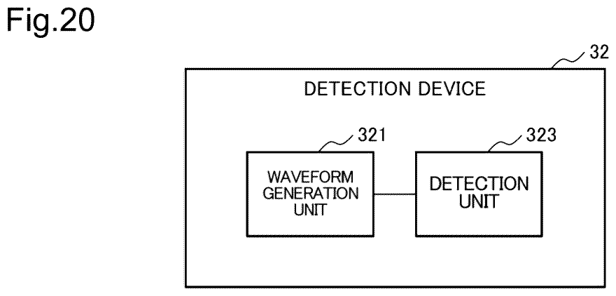

Next, a detection device according to a third example embodiment will be described with reference to the drawings. The detection device of the present example embodiment has a configuration in which the detection device of each example embodiment is simplified. FIG. 20 is a block diagram illustrating an example of a configuration of a detection device 32 of the present example embodiment. The detection device 32 includes a waveform generation unit 321 and a detection unit 323.

The waveform generation unit 321 generates a gait waveform using sensor data related to the motion of the foot. The detection unit 323 detects the gait event from the gait waveform based on the condition set for each of the angle, the angular velocity, and the acceleration in the sagittal plane.

According to the detection device of the present example embodiment, since the gait event is detected from the gait waveform based on the condition set for each of the angle, the angular velocity, and the acceleration in the sagittal plane, the gait event can be detected based on the gait waveform not only for a gait of a healthy person but also for a gait of a person with physical disability.

(Hardware)

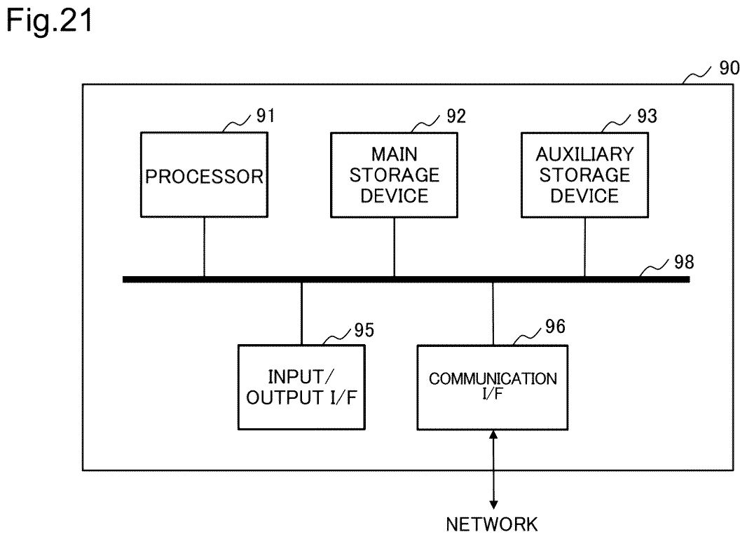

A hardware configuration for performing the processing of the detection device according to each example embodiment of the present disclosure will be described using an information processing device 90 of FIG. 21 as an example. The information processing device 90 in FIG. 21 is a configuration example for performing processing of the detection device of each example embodiment, and does not limit the scope of the present disclosure.

As illustrated in FIG. 21, the information processing device 90 includes a processor 91, a main storage device 92, an auxiliary storage device 93, an input/output interface 95, and a communication interface 96. In FIG. 21 the interface is abbreviated as an interface (I/F). The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops the program stored in the auxiliary storage device 93 or the like in the main storage device 92 and executes the developed program. In the present example embodiment, a software program installed in the information processing device 90 may be used. The processor 91 executes processing by the detection device according to the present example embodiment.

The main storage device 92 has an area in which a program is developed. The main storage device 92 may be a volatile memory such as a dynamic random access memory (DRAM). A nonvolatile memory such as a magnetoresistive random access memory (MRAM) may be configured and added as the main storage device 92.

The auxiliary storage device 93 stores various pieces of data. The auxiliary storage device 93 includes a local disk such as a hard disk or a flash memory. Various pieces of data may be stored in the main storage device 92, and the auxiliary storage device 93 may be omitted.

The input/output interface 95 is an interface that connects the information processing device 90 with a peripheral device. The communication interface 96 is an interface that connects to an external system or a device through a network such as the Internet or an intranet in accordance with a standard or a specification. The input/output interface and the communication interface 96 may be shared as an interface connected to an external device.

An input device such as a keyboard, a mouse, or a touch panel may be connected to the information processing device 90 as necessary. These input devices are used to input information and settings. When the touch panel is used as the input device, the display screen of the display device may also serve as the interface of the input device. Data communication between the processor 91 and the input device may be mediated by the input/output interface 95.

The information processing device 90 may be provided with a display device that displays information. In a case where a display device is provided, the information processing device 90 preferably includes a display control device (not illustrated) that controls display of the display device. The display device may be connected to the information processing device 90 via the input/output interface 95.

The information processing device 90 may be provided with a drive device. The drive device mediates reading of data and a program from the recording medium, writing of a processing result of the information processing device 90 to the recording medium, and the like between the processor 91 and the recording medium (program recording medium). The drive device may be connected to the information processing device 90 via the input/output interface 95.

The above is an example of a hardware configuration for enabling the detection device according to each example embodiment of the present invention. The hardware configuration of FIG. 21 is an example of a hardware configuration for executing arithmetic processing of the detection device according to each example embodiment, and does not limit the scope of the present invention. A program for causing a computer to execute processing related to the detection device according to each example embodiment is also included in the scope of the present invention. A program recording medium in which the program according to each example embodiment is recorded is also included in the scope of the present invention. The recording medium can be achieved by, for example, an optical recording medium such as a compact disc (CD) or a digital versatile disc (DVD). The recording medium may be achieved by a semiconductor recording medium such as a Universal Serial Bus (USB) memory or a secure digital (SD) card, a magnetic recording medium such as a flexible disk, or another recording medium. In a case where the program executed by the processor is recorded in the recording medium, the recording medium is a program recording medium.

The components of the detection device of each example embodiment can be combined in any manner. The components of the detection device of each example embodiment may be achieved by software or may be achieved by a circuit.

While the present invention is described with reference to example embodiments thereof, the present invention is not limited to these example embodiments. Various modifications that can be understood by those skilled in the art can be made to the configuration and details of the present invention within the scope of the present invention.

### REFERENCE SIGNS LIST

1, 2 detection system
11, 21 data acquisition device
12, 22, 32 detection device
111 acceleration sensor
112 angular velocity sensor
113 control unit
115 data transmission unit
121, 221, 321 waveform generation unit
123, 223, 323 detection unit
225 determination unit

What is claimed is:

1. A detection device comprising:
at least one memory storing instructions; and
at least one processor connected to the at least one memory and configured to execute the instructions to:
generate a gait waveform using sensor data related to a motion of a foot; and
detect a gait event from the gait waveform based on a condition set for each of an angle, an angular velocity, and an acceleration in a sagittal plane.

2. The detection device according to claim 1, wherein
the at least one processor is configured to execute the instructions to
set a window for a predetermined time for a gait waveform represented by each of an angle, an angular velocity, and an acceleration in the sagittal plane, and
detect the gait event by sliding the window in a time direction.

3. The detection device according to claim 2, wherein
the at least one processor is configured to execute the instructions to
detect, based on a first detection condition for detecting a peak candidate based on a magnitude relationship between an angle value at timing of both ends of the window and a maximum angle inside the window in a gait waveform represented by an angle in the sagittal plane and a first determination condition for determining whether the peak candidate is a peak, the peak from a gait waveform represented by an angle in the sagittal plane.

4. The detection device according to claim 3, wherein
is configured to the at least one processor is configured to execute the instructions to
determine, based on a second detection condition for detecting a location where an amount of change in an angular velocity inside the window is steep in a gait waveform represented by an angular velocity in the sagittal plane, and a second determination condition for determining which of a plantarflexion peak and a dorsiflexion peak a location where an amount of change in an angular velocity inside the window is steep is associated with based on a magnitude relationship between a value of an angular velocity at a timing of both ends of the window and a maximum angular velocity inside the window, which of the plantarflexion peak and the dorsiflexion peak the peak detected from a gait waveform represented by an angle in the sagittal plane is associated with.

5. The detection device according to claim 3, wherein
the detection unit the at least one processor is configured to execute the instructions to
determine, based on a second detection condition for detecting a location where an amount of change in an angular velocity inside the window is steep in a gait waveform represented by an angular velocity in the sagittal plane and a third determination condition for determining which of a plantarflexion peak and a dorsiflexion peak the peak is associated with based on a value of an acceleration in a traveling direction in the sagittal plane at a timing of the peak detected inside the window, which of the plantarflexion peak and the dorsiflexion peak the peak detected from a gait waveform represented by an angle in the sagittal plane is associated with.

6. The detection device according to claim 4, wherein
the at least one processor is configured to execute the instructions to determine a gait state based on the peak detected by the detection unit.

7. The detection device according to claim 6, wherein
the at least one processor is configured to execute the instructions to
determine a period between the plantarflexion peak and the dorsiflexion peak that are consecutive as a swing phase,
determine a period between the dorsiflexion peak and the plantarflexion peak that are consecutive as a stance phase, and
output information indicating which of the swing phase and the stance phase a time of the gait waveform generated by the waveform generation unit is associated with.

8. A detection system comprising:
the detection device according to claim 1; and
a data acquisition device that is disposed on footwear worn by a user that is a target for which a gait waveform is to be measured, measures a spatial acceleration and a spatial angular velocity according to a gait of the user, generates sensor data based on the measured spatial acceleration and the measured spatial angular velocity, and transmits the generated sensor data to the detection device.

9. A detection method executed by a computer, the method comprising:
generating a gait waveform using sensor data related to a motion of a foot; and
detecting a gait event from the gait waveform based on a first condition, a second condition, and a third condition set for an angle, an angular velocity, and an acceleration, respectively, in a sagittal plane.

10. A non-transitory program recording medium recording a program for causing a computer to execute:
generating a gait waveform using sensor data related to a motion of a foot; and
detecting a gait event from the gait waveform based on a first condition, a second condition, and a third condition set for an angle, an angular velocity, and an acceleration, respectively, in a sagittal plane.
